(19) United States
(12) Patent Application Publication
(10) Pub. No.: US 2024/0041423 A1
(43) Pub. Date: Feb. 8, 2024

(54) ULTRASOUND DEVICE TRACKING SYSTEM

(71) Applicants: SORBONNE UNIVERSITE, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MÉDICALE), Paris (FR)

(72) Inventors: Antoine COUDERT, Paris (FR); Olivier COUTURE, Issy-les-Moulineaux (FR); Vincent HINGOT, Paris (FR)

(73) Assignees: SORBONNE UNIVERSITE, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MÉDICALE), Paris (FR)

(21) Appl. No.: 18/256,706

(22) PCT Filed: Dec. 10, 2021
(86) PCT No.: PCT/EP2021/085222

§ 371 Date: Jun. 9, 2023

(30) Foreign Application Priority Data
Dec. 11, 2020 (EP) 20306553.7

(51) Int. Cl. A61B 8/08; A61B 8/00

(52) U.S. Cl. CPC A61B 8/085; A61B 8/0808; A61B 8/0891; A61B 8/4227; A61B 8/4254; A61B 8/4477; A61B 8/5215

(57) ABSTRACT

A device tracking system for monitoring a target body structure and localizing a device inside the target body structure. The device tracking system includes: a control unit, a device having one steering element to be handled outside the target body structure and one steerable element to be introduced inside the target body structure, one probe to be removably secured to the patient, and one tracker to be secured to the steerable element. The control unit stores one ultrasound image of the target body structure. The probe and the tracker communicate, and the control unit is able to localize, in real time, the steerable element. The control unit displays, the stored ultrasound image and displays, in real time, the localisation of the at steerable element on the at least one ultrasound image.

### FIELD

The present invention relates to ultrasound imaging, in particular ULM imaging and ultrasound communication used in device localization inside a target body structure of a patient.

### BACKGROUND

Inspired by single molecule localization microscopy in optics which revolutionized cell imaging, Ultrasound Localisation Microscopy (ULM) has recently begun to revolutionize biomedical ultrasound imaging.

It is well known from the state of the art that the resolution in B-mode imaging and Doppler imaging is limited both axially and laterally by the wavelength. The ultimate limit is set by the Rayleigh criterion. ULM overcomes this diffraction barrier and enables to push the resolution-attenuation trade-off further.

The core idea of ULM is knowingly to introduce sparse punctual sources in the medium being imaged, to highlight specific parts. These sources are gas microbubbles, more precisely millions of microbubbles, also called contrast agents. Thanks to these microbubbles, the vascular system is resolved under the diffraction barrier. A super-resolved image (or ULM image) is constructed by localizing each bubble centre separately and accumulating their positions to recover the vessel's network, several times smaller than the wavelength. The position of these microbubbles can be found with a precision greater than the ultrasonic wavelength by using “ULM localization” techniques (for example but not limited to techniques like weighted average, interpolation, radial symmetry, gaussian fitting). The use of microbubbles (with a diameter ranging from 1 to 3 μm), thanks to their high compressibility, allows the imaging system to outperform accuracy limitations due to the classical wave diffraction theory which is around half of the wavelength and to bypass the usual compromise to be found between wave penetration (favoured in the low way frequency range) and image resolution (favoured in the high way frequency range). This enables to visualize details which remain invisible on images built by conventional echography, Doppler echography in particular.

In particular regarding brain vascularization, this technology enables the creation of highly precise images enabling a precise mapping of a patient's brain vascular system. In case of a stroke, in the particular case of a thrombectomy operation, this mapping could highly improve the ability of a surgeon to navigate a catheter tip inside the brain's blood vessel. It is well known by any person skilled in the art that just after a stroke, a recent occlusion can be reopened without surgery by thrombolysis, thrombo-aspiration or mechanical thrombectomy, for example. All those techniques necessitate, in at least one of their implementation options, the surgeon to navigate a catheter tip through the brains vascular system in order to reach the occlusion point. Nowadays, the surgeon visualizes the catheter by means of fluoroscopy, a 2D x-ray-based technique that does not allow the 3D tracking of the catheter. This technique leads to a 2-dimensional imaging obtained by means of X rays and can be associated to 3 dimensional MRI data. Generally, the surgeon is shown one or two plane fluoroscopy images, which are x-ray projection images, meanwhile (s)he has access to an MRI scan on another screen. The placement of the catheter thus implies to inject X-Ray contrast agents which may comprise some health damaging elements. This leads to well-known localisation and navigation difficulties, as the surgeon lacks a 3D mapping of the brain vascularization, allowing a 3D navigation plan. This is even more important in the brain (with regards to other parts of the body) as the brain includes lots of very tortuous blood vessels which are not well cut out on 2D imaging.

The aim of the current invention is thus to provide an accurate visual localisation of a device inside a target body structure on a 3D mapping or image of the target body structure, in order to facilitate the navigation of a device inside this target body part.

### SUMMARY

This invention thus relates to a device tracking system configured to monitor a target body structure of a patient and localizing a device inside said target body structure, the tracking system comprising:
a control unit with a memory,
a device comprising:
   at least one steering element configured to be handled outside the target body structure,
   at least one steerable element configured to be introduced inside the target body structure, the steerable element and the steering element being physically connected by at least one connection element,
at least one probe configured to be brought in contact with a securing body part of the patient, the securing body part surrounding at least partially the target body structure,
at least one tracker configured to be secured to the steerable element of the device, wherein the memory is configured to store at least one ultrasound image of the target body structure,
   wherein the at least one probe and the at least one tracker are configurated to communicate by means of ultrasounds, the control unit being thus configured to localize, in real time, the steerable element inside the target body structure,
   wherein the ultrasound image (29) is an ULM image,
   wherein the control unit is further configured to display, on a screen, the at least one stored ultrasound image and display, in real time, the localisation of the at steerable element on said at least one ultrasound image.

This way, the solution enables to reach the here-above mentioned objective. Especially, the use of the tracking system enables an improved precision and thus a quicker and safer interventional response from the surgeon, in particular after a stroke, in order to minimize the impact of said stroke, particularly regarding occlusions.

The stroke scanner according to the invention may comprises one or several of the following features, taken separately from each other or combined with each other:
the control unit is configurated to localize the steerable element by means of ULM localization,
the at least one probe may comprise at least one ultrasound transducer and the at least one tracker may comprise at least one ultrasound sensor,
the at least one probe is configured to be removably secured to the securing body part of the patient, the securing body part surrounding at least partially the target body structure,
the at least one tracker may comprise an object strongly reflecting ultrasounds waves,
the at least one tracker may comprise at least one ultrasound transducer and the at least one probe may comprise at least one ultrasound sensor,
the at least one steering element may be configured to be handled manually,
the at least one steerable element may measure between 0.1 and 3 mm,
the device may be a catheter, the at least one steerable element may be a catheter tip, and the at least one steering element may be a catheter handle,
the memory of the control unit may be configured to store a succession of ultrasound image of the target body structure, each new ultrasound image may replace the prior one,
the ultrasound image acquisition may be done in real time, a new ultrasound image acquisition being launched as soon the prior ultrasound image acquisition is terminated, each new ultrasound image replacing the prior one as soon its acquisition is terminated,
the target body structure may be the vascular system of the brain,
the control unit is able to localize the steerable element with a better precision than half the size of the wavelength of the ultrasound used to perform the localization.

The system may further be for use in thrombectomy.

The invention also relates to a device tracking and localization method implemented by means of the system according to any one of the preceding claims, wherein the method enables, at the same time:
the visualization, on a screen, of an ultrasound image of a target body structure of a patient,
the real time tracking of the steering element,
the real time localization of said steering element inside the target body structure,
the real time display, on the screen (30), within the displayed ultrasound image of said steering element localization.

### BRIEF DESCRIPTION OF THE DRAWINGS

FIG. 1 is a ULM image of a target body structure,

FIG. 2 is a schematic view of the one embodiment of the system according to the present invention,

FIG. 3 is a schematic view of the device inserted in the target body structure according to the present invention,

FIG. 4 is an example of a visualization obtained by means of the device according to the present invention.

### DETAILED DESCRIPTION

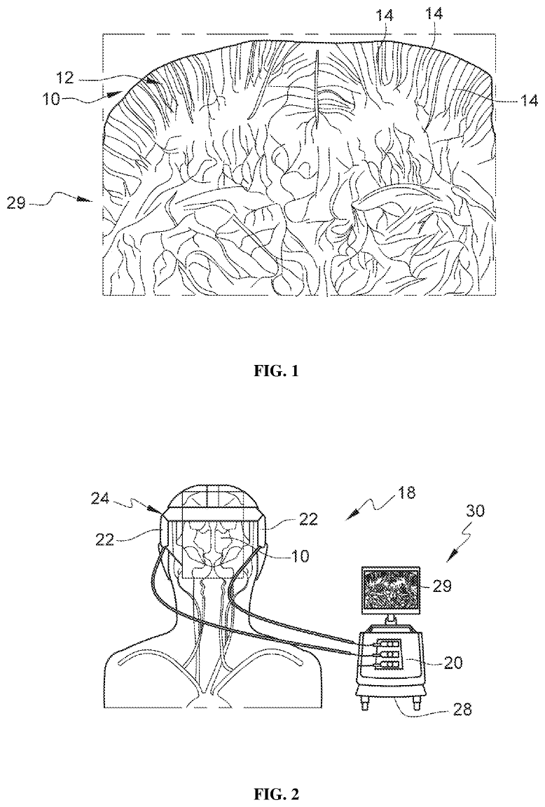

As can be seen on FIG. 1, a typical target body structure 10 like a brain vascular system 12 counts an amazingly high number of blood vessels 14. It is well known from any person skilled in the art that the normal function of the brain's control centres (for example, but this is true for any organ in the body) depends upon adequate oxygen and nutrients supply through a dense network of blood vessels 14. Blood is usually supplied to the brain, face, and scalp via two major sets of vessels 14: the right and left common carotid arteries and the right and left vertebral arteries. The common carotid arteries are well known to display two divisions:
the external carotid arteries supply the face and scalp with blood,
the internal carotid arteries supply blood to most of the anterior portion of the cerebrum,
the vertebrobasilar arteries supply the posterior two-fifths of the cerebrum, part of the cerebellum, and the brain stem.

Any decrease in the flow of blood through one of the internal carotid arteries brings about some impairment in the function of the frontal lobes. This impairment may result in numbness, weakness, or paralysis on the side of the body opposite to the obstruction of the artery. Even worse, occlusion of one of the vertebral arteries can cause many serious consequences, ranging from blindness to paralysis, or death, in millions of cases per year.

It is therefore essential to intervene as soon as possible to provide blood flow to these ischemic areas. One treatment is thrombolysis (tPA), which can only be injected within the first few hours after the onset of symptoms.

Another option is thrombectomy, where a catheter is introduced in the vascular system and inserted up to the thrombus in the artery to mechanically remove it.

Occlusions are thus well known to be treated by means of a device 15, for example a catheter, introduced in the damaged blood vessel 14. Regarding the present invention, such a device 15 comprise at least one steerable element 16 and at least one steering element 17. The steering element 17 is handled from outside the target body structure 10 by a surgeon or a robotic device, for example. This steering element 17 enables the mechanical steering and positioning of the steerable element 16 which is configured to be introduced inside the target body structure 10. The steering element 17 thus enables the direct manually or robotic steering of the steerable element 16. The steerable element 16 and the steering element 17 are thus physically connected by at least one physical connection element 18. The steerable element 16, each steering element 17 and the connection element 18 can all be produced in one piece or can be removable secured to each other. In the case where said device 15 is a catheter, the steerable element 16 is the catheter tip, the steering element 17 is a catheter handle and the connection elements 18 form the catheter body. In case the device 15 is a catheter, the steerable element 16 is introduced through the intra-femoral artery inside the patient's brain vascular system 12 and manually navigated, by means of the steering element 17, through said vascular system 12 until the occlusion point of the damaged vessel 14 is reached. In order to help navigating the steerable element 16 (in the present example, the catheter tip) through the vascular system 12, the surgeon needs visual help. This visual help is usually displayed on a screen and necessitates to monitor the inserted steerable element 16.

According to the present invention, and as can be seen on FIG. 2, this monitoring is done by means of a tracking system 19 configured to monitor the target body structure of the patient. This system 19 according to the current invention comprises:
a control unit 20 with a memory,
at least one probe 22, more particularly an ultrasound probe, configured to be removably secured to a securing body part 24 of the patient, the securing body part 24 surrounding at least partially the target body structure 10,
at least one tracker 26 configured to be secured to the steerable element 15 and thus configured to be introduced inside the target body structure 10.

The control unit 20 is an emission/reception system which transforms electric impulses into acoustic impulses (and vice versa) in order to enable the acoustic characteristics of a given environment, in this case, the target body structure 10.

In the embodiment illustrated on FIG. 2, the system 19 includes two probes 22, each secured to a temple of the patient. The securing body part 24 in this embodiment is thus the forehead of the patient. In some other embodiments, not shown, there could be more probes, secured on different securing body parts 24. In some other embodiments, a probe might be handheld.

The probe 22 are brought in contact with the securing body part 24. Even in case a gel is spread between the securing body part 24 and the probe 22, it is considered that the probe is in contact with the securing body part 24. The probe could be manually handled. In another embodiment, the probes 22 are secured to the securing body part 24 for example by means of a helmet or an elastic holder, as can be seen on FIG. 2. The probes 22 have to be easily, quick and removably secured to the securing body part 24 of the patient. In some embodiments, each probe 22 comprises at least one ultrasound transducer, for example a piezo-electric transducer. In some other embodiments, each probe 22 comprises at least one ultrasound sensor, preferably three sensors in order to be able to localize the tracker 26 by means of triangulation. Each probe 22 is in real time communication with the control unit 20.

In the current application, the term “transducer” is used synonymously as “emitter” and the term “sensor” is used synonymously as “receptor”.

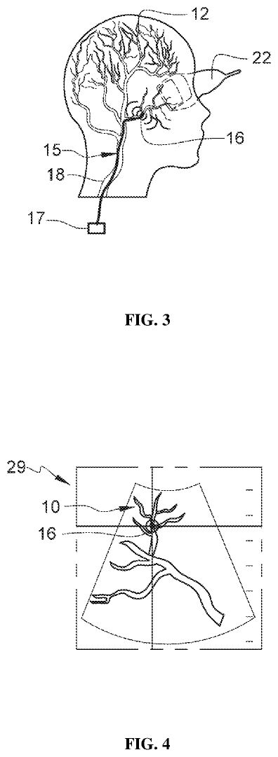

According to the embodiment illustrated on FIG. 3, the at least one tracker 26 is secured to the steerable element 16 of the device 15, which steerable element 16 can be manually introduced inside the target body structure 10 by means of the steering element 17 and the connection elements 18. This kind of steerable elements 16 usually measures less than 1 cm in diameter, more particularly between 0.1 and 3 mm in diameter. In some embodiments, each at least one second tracker 26 comprises at least one ultrasound transducer, for example a piezo-electric transducer. In some other embodiments, the at least one second tracker 26 comprises at least one ultrasound sensor, for example a strongly reflective object.

In every embodiment, the probe and the tracker 22, 26 communicate by means of ultrasounds, regardless of which ones are the transducer(s) or the sensor(s). In the case of the tracker 26 used as sensor, there is no need to generate high-voltages inside the device 15. The device 15 is thus a passive device. In the case of the tracker 26 used as a transducer (emitter), it allows better signal-to-noise ratio in the positioning. The information collected by each probe 22 from the at least one tracker 26 is sent, in real time, to the control unit 20. The control unit 20 is thus able to localize, in real time, the at least one tracker inside 26 the target body structure 10. Depending on the ultrasound technology used, the control unit 20 is able to localize the steerable element with a better precision than half the size of the wavelength of the ultrasound used to perform the localization.

The control unit 20 further comprises a memory 28 configured to store at least one ultrasound image 29, for example an ULM image 29 of the target body structure 10, like for example the image illustrated on FIG. 1. This ultrasound image 29 provides a 3D anatomical mapping of the target body structure 10. In case the ultrasound image 29 is an ULM image 29, this ULM image 29 provides a very precise 3D mapping of the target body structure 10 of the patient. In order to obtain an ULM image of a target body structure 10 like the brain vascular system 12, microbubbles are injected in the patient. Many 3D transcranial images are acquired. Microbubbles are localized and within a few minutes, a 3D ULM image is obtained.

This ultrasound image 29 can be obtained prior to the monitoring of the target body structure 10 by the tracking system 19 or during the monitoring of the target body structure 10 by the system 19. In some embodiments, the memory 28 of the control unit 20 can store several ultrasound images 29 of the target body structure 10. The memory 28 can thus store a succession of ultrasound image 29 of the target body structure 10. In some embodiments, in order to reduce storage energy, each new ultrasound image 29 replaces the prior one inside the memory 28. In order to increase the precision and accuracy of the mapping of the target body structure 10 during its monitoring by the system 19, the ultrasound image acquisition is done in real time. This provides a real time mapping of the target body structure 10 and enables to take quick structure changes into consideration. For example, the case where the device 15 is a catheter, navigating the catheter through the brain vascular system 12 deforms the blood vessels 14 to a certain degree and may shift some curvature or angle with regards to the ultrasound image 29 acquired before inserting the device 15 into the target body structure 10. This real time mapping occurs in that a new ultrasound image acquisition is launched, by the control unit 20 as soon the prior ultrasound image acquisition is terminated, each new ultrasound image 29 thus replacing the prior one as soon its acquisition is terminated.

The control unit 20 is also designed to display, on a screen 30, each acquired and/or stored ultrasound image 29. This is illustrated on FIG. 2.

By combining the real time information obtained from each first probe 22 regarding the at least one tracker 26 and the information of the stored ultrasound image 29, the control unit 20 is able to display, in real time, the localisation of the at least one tracker 26 on said ultrasound image 29, as shown on FIG. 4. This enables the surgeon to know, precisely, where the device 15, and more particularly the steerable element 16 of the device 15 is and how and to where it is to be manipulated (by means of the steering element 17) in order to reach the occlusion point (for example).

The system 19 thus enables to implement a device 15 tracking and localization method, wherein the method enables:
the visualization, on the screen 30, of at least one ultrasound image 29, for example an ULM image 29, of the target body structure 10 of a patient,
the real time tracking of the steerable element 16 of the device 15,
the real time localization of said steerable element 16 inside the target body structure 10,
the real time display, on the screen, 30 within the ultrasound image 29 of said steerable element 16 localization.

1-13. (canceled)

14. A device tracking system configured to monitor a target body structure of a patient and localizing a device inside said target body structure, the tracking system comprising:
a control unit with a memory,
a device comprising:
at least one steering element configured to be handled outside the target body structure,
at least one steerable element configured to be introduced inside the target body structure, the steerable element and the steering element being physically connected by at least one connection element,
at least one probe configured to be brought in contact with a securing body part of the patient, the securing body part surrounding at least partially the target body structure,
at least one tracker configured to be secured to the steerable element of the device,
wherein the memory is configured to store at least one ultrasound image of the target body structure,
wherein the at least one probe and the at least one tracker is configurated to communicate by means of ultrasounds, the control unit being thus configurated to localize, in real time, the steerable element inside the target body structure,
wherein the ultrasound image is an ULM image,
wherein the control unit is further configured to display, on a screen, the at least one stored ultrasound image and display, in real time, the localisation of the steerable element on said at least one ultrasound image.

15. The system according to claim 14, wherein the control unit is configurated to localize the steerable element by means of ULM localization.

16. The system according to claim 14, wherein the at least one probe is configured to be removably secured to the securing body part of the patient, the securing body part surrounding at least partially the target body structure.

17. The system according to claim 14, wherein the at least one probe comprises at least one ultrasound transducer and the at least one tracker comprises at least one ultrasound sensor.

18. The system according to claim 17, wherein the at least one tracker comprising an object strongly reflecting ultrasounds waves.

19. The system according to claim 14, wherein the at least one tracker comprises at least one ultrasound transducer and the at least one probe comprises at least one ultrasound sensor.

20. The system according to claim 14, wherein the at least one steering element is configured to be handled manually.

21. The system according to claim 20, wherein the at least one steerable element measures between 0.5 and 3 mm.

22. The system according to claim 14, wherein the device is a catheter, the at least one steerable element is a catheter tip, and the at least one steering element is a catheter handle.

23. The system according to claim 14, wherein the memory of the control unit is configured to store a succession of ultrasound image of the target body structure, each new ultrasound image replacing the prior one.

24. The system according to claim 23, wherein the ultrasound image acquisition is done in real time, a new ultrasound image acquisition being launched as soon the prior ultrasound image acquisition is terminated, each new ultrasound image replacing the prior one as soon its acquisition is terminated.

25. The system according to claim 14, wherein the target body structure is the vascular system of the brain.

26. The system according to claim 14, wherein the system is able to localize the steerable element with a better precision than half the size of the wavelength of the ultrasound used to perform the localization.

27. The system according to claim 14, wherein the system is for use in thrombectomy.
